(19) United States
(12) Patent Application Publication
(10) Pub. No.: US 2025/0312431 A1
(43) Pub. Date: Oct. 9, 2025

(54) VACCINES AGAINST MORITELLA VISCOSA

(71) Applicant: Zoetis Services LLC, Parsippany, NJ (US)

(72) Inventors: Anette FUREVIK, Askoy (NO); Lars Gaute JORGENSEN, Tromso (NO); Ane Sandtro LUNHEIM, Trondheim (NO); Monica Gausdal TINGBO, Oslo (NO); Siv Haugen TUNHEIM, Overhalla (NO)

(21) Appl. No.: 18/864,723

(22) PCT Filed: May 12, 2023
(86) PCT No.: PCT/US2023/066908

§ 371 Date: Nov. 11, 2024

Related U.S. Application Data
(60) Provisional application No. 63/342,250, filed on May 16, 2022.

(51) Int. Cl. A61K 39/02; A61K 39/00; A61P 37/04

(52) U.S. Cl. CPC A61K 39/0208; A61P 37/04; A61K 2039/521; A61K 2039/552

(57) ABSTRACT

The invention provides vaccines against emerging isolates of M. viscosa and methods of using said vaccines

### FIELD OF THE INVENTION

This invention is generally in the field of aquaculture vaccines.

### BACKGROUND

Winter ulcer disease affects both Atlantic salmon (Salmo salar) and rainbow trout (Oncorhynchus mykiss) and results in increased mortality rates as well as major economical losses due to downgrading of fish at slaughter.

Moritella viscosa (formally Vibrio viscosus) is the main aetiological agent of winter ulcer disease (Løvoll et al., 2009; Tunsjø et al., 2009; Björnsson et al., 2011; Karlsen et al., 2017a; Karlsen et al., 2017b). It is a gram-negative, psychrophilic, facultative anaerobic bacterium capable of both fermentative and respiratory metabolisms (Gudmundsdóttir and Björnsdóttir, 2007; Tunsjø et al., 2009; Björnsson et al., 2011). It is oxidase and catalase positive, requiring salt for growth; colonies are yellowish-translucent and generally viscous (Gudmundsdóttir and Björnsdóttir, 2007), although non-viscous M viscosa strains have also been isolated in the recent years.

Vaccines exist that protect against classic viscous strains of M. viscosa. However, current commercial vaccines are not effective against emerging strains that are both genotypically and/or phenotypically different from the classic strains. These emerging strains can be classified as variant, based on gyrB sequence, and classic non-viscous strains based on the non-viscous appearance after being cultured in agar plates, in contrast to the classic viscous M. viscosa strains that present adherent colonies forming viscous threads when manipulated with a loop.

Accordingly, new vaccines and methods are needed to effectively protect against the emerging strains as well as classic viscous strains.

### SUMMARY OF INVENTION

This disclosure addresses these and other needs by providing, in the firth aspect, a vaccine comprising an antigenic M. viscosa component, said antigenic M. viscosa component comprising an antigen derived from a classic non-viscous M. viscosa strain, for use in protecting fish against infection caused by variant M. viscosa.

The vaccine according to the first aspect of the invention may be used in protecting fish against infection caused by variant M. viscosa and classic non-viscous M. viscosa.

Also disclosed is a vaccine according to the first aspect of the invention, wherein the antigenic component consists essentially of, or consists of, the antigen derived from the antigen derived from the classic non-viscous M. viscosa strain.

Also disclosed is a vaccine according to the first aspect of the invention, wherein the antigenic component consists essentially of, or consists of, the antigen derived from the classic non-viscous M. viscosa strain and, optionally, an antigen derived from a classic viscous M. viscosa strain.

The vaccine according to the first aspect of the invention may be co-administered with a second vaccine, wherein the second vaccine of this first aspect comprises an antigen derived from a classic viscous M. viscosa strain. Preferably, the second vaccine does not contain an antigen derived from a variant strain of M. viscosa.

In the vaccine according to the first aspect of the invention, the antigen derived from the classic non-viscous M. viscosa strain is an inactivated preparation of the classic non-viscous M. viscosa strain. If the antigen derived from the classic viscous M. viscosa strain is present in the vaccine according to the first aspect, or in the second vaccine of the first aspect, said antigen may be in the form of an inactivated preparation of the classic viscous M. viscosa strain.

In the second aspect, this disclosure provides a vaccine comprising an antigenic M. viscosa component, said antigenic M. viscosa component comprising an antigen derived from a variant M. viscosa strain, for use in protecting fish against infection caused by classic non-viscous M. viscosa.

The vaccine according to the second aspect of the invention may be used in protecting fish against infection caused by variant M. viscosa and classic non-viscous M. viscosa.

Also disclosed is a vaccine according to the second aspect of the invention, wherein the antigenic component consists essentially of, or consists of, the antigen derived from the antigen derived from the variant M. viscosa strain.

Also disclosed is a vaccine according to the second aspect of the invention, wherein the antigenic component consists essentially of, or consists of, the antigen derived from the variant M. viscosa strain and, optionally, an antigen derived from a classic viscous M. viscosa strain.

The vaccine according to the second aspect of the invention may be co-administered with a second vaccine of the second aspect, wherein the second vaccine comprises an antigen derived from a classic viscous M. viscosa strain. Preferably, the second vaccine of the second aspect does not contain an antigen derived from a classic non-viscous strain of M. viscosa.

Also disclosed in this second aspect is a vaccine wherein the antigen derived from the variant M. viscosa strain is an inactivated preparation of the variant M. viscosa strain. If the antigen derived from the classic viscous M. viscosa strain is present in the vaccine according to the second aspect, or in the second vaccine of the second aspect, said antigen may be in the form of an inactivated preparation of the classic viscous M. viscosa strain.

In the third aspect, this disclosure provides a vaccine comprising an antigenic M. viscosa component, said antigenic M. viscosa component comprising an antigen derived from a variant M. viscosa strain, for use in protecting fish against infection caused by a classic non-viscous M. viscosa and classic viscous M. viscosa.

The vaccine according to this third aspect may be used in protecting fish against infection caused by variant M. viscosa, classic non-viscous M. viscosa and classic viscous M. viscosa.

The vaccine according to this third aspect of the invention does not include an antigen derived from a classic non-viscous M. viscosa strain and does not include an antigen derived from a classic viscous M. viscosa strain. In certain embodiments, the M. viscosa component of the vaccine according to this third aspect of the invention consists essentially or consists of the antigen derived from the variant M. viscosa strain.

In the vaccines according to this third aspect of the invention, the antigen derived from the variant M. viscosa strain is an inactivated preparation of said variant M. viscosa strain.

In the fourth aspect, the disclosure provides a vaccine comprising an antigenic M. viscosa component, said antigenic M. viscosa component comprising an antigen derived from a classic non-viscous M. viscosa strain, for use in protecting fish against infection caused by variant M. viscosa and classic viscous M. viscosa.

The vaccine according to this fourth aspect may be used in protecting fish against infection caused by variant M. viscosa, classic non-viscous M. viscosa and classic viscous M. viscosa.

The vaccine according to this fourth aspect of the invention does not include an antigen derived from a variant M. viscosa strain and does not include an antigen derived from a classic viscous M. viscosa strain. In certain embodiment, the M. viscosa component of the vaccine according to this fourth aspect of the invention consists essentially or consists of the antigen derived from the classic non-viscous M. viscosa strain.

In the vaccines according to this fourth aspect of the invention, the antigen derived from the classic non-viscous M. viscosa strain is an inactivated preparation of said classic non-viscous M. viscosa strain.

The compositions according to the first, the second, the third, and the fourth aspect of the invention further contain non-M. viscosa antigens. In certain embodiments, said one or more non-M. viscosa antigens are selected from the group consisting of IPNV, ISAV, SPDV, Aeromonas salmonicida, Vibrio anguillarum O1, O2, Vibrio (Aliivibrio) salmonicida, Yersinia ruckeri 01.

The compositions according to the first, the second, the third, and the fourth aspect of the invention are provided as water-in-oil emulsions.

Any compositions described above preferably are used in salmonids, most preferably, Atlantic salmon (Salmo salar). In certain embodiments, the weight of said fish is about 15-200 grams at the time of vaccination.

The compositions disclosed herein are suitable for protecting said fish against infection comprises a reduction or an elimination of at least one symptom of M. viscosa. In certain embodiments, the at least one symptom is mortality.

### BRIEF DESCRIPTION OF THE DRAWINGS

FIG. 1 is a photograph of Western Blot demonstrating that different antibodies recognize classic viscous M. viscosa on one hand and classic non-viscous M. viscosa and variant M. viscosa on the other.

FIG. 2 illustrates the cumulative survival of fish vaccinated with classic viscous, classic non-viscous and variant M. viscosa after the challenge with classic non-viscous M. viscosa.

FIG. 3 illustrates the cumulative survival of fish vaccinated with classic viscous, classic non-viscous and variant M. viscosa after the challenge with variant M. viscosa.

FIG. 4 illustrates the cumulative survival of fish vaccinated with classic viscous and variant M. viscosa after the challenge with classic viscous M. viscosa.

### DETAILED DESCRIPTION

For a better understanding of the invention, the following non-limiting definitions are provided:

The term “about” or “approximately,” when used in connection with a measurable numerical variable, refers to the indicated value of the variable and to all values of the variable that are within the experimental error of the indicated value (e.g., within the 95% confidence interval for the mean) or within 10 percent of the indicated value, whichever is greater.

The term “antigenic M. viscosa component” refers to one or more antigens derived from M. viscosa, including classic viscous strains, classic non-viscous strains and variant strains.

“Antigen derived from” a pathogen, including classic M. viscosa, classic non-viscous M. viscosa, and variant M. viscosa refers to the inactivated preparation of the desired M. viscosa subtype, as well as whole-bacterial extract and fractions of the extract including without limitations membrane/cell wall extract.

“Classic M. viscosa” also referred to as “viscous M. viscosa” or “classic viscous M. viscosa” refers to M. viscosa strains that form viscous adherent colonies when cultured on blood agar at 15° C. for 48 hours, with NaCl concentration below 2.5%. M. viscosa usually forms greyish colonies. When the colonies are manipulated with the loop, the colonies of classic viscous M. viscosa form viscous mucous threads.

“Classic non-viscous M. viscosa” refers to M. viscosa strains classified as classic based on gyrB sequences but these strains do not form viscous adherent colonies when cultured on blood agar at 15° C. for 48 hours, with NaCl concentration below 2.5%.

Classic isolates (both viscous and non-viscous) of M. viscosa have a conserved sequence in their respective gyrB genes. Accordingly, classic isolates are the isolates that contain, in their respective gyrB sequences, a subsequence that is at least 96% identical to SEQ ID NO: 1 (for example, at least 97% or at least 98% identical).

Two or more vaccines are “co-administered” if they are administered within 15 minutes of each other. Preferably, said two or more vaccines are administered within 10 minutes, or within 5 minutes, or within 4 minutes, or within 3 minutes, or within 2 minutes, or within 1 minutes of each other.

“M. viscosa” which is not preceded by classic, or variant, or non-viscous encompasses all three subtypes of M. viscosa.

The term “pharmaceutically acceptable” refers to substances, which are within the scope of sound medical judgment, suitable for use in contact with the tissues of subjects without undue toxicity, irritation, allergic response, and the like, commensurate with a reasonable benefit-to-risk ratio, and effective for their intended use.

The term “subject” refers to fish for which the administration of an adjuvant composition is desired.

The phrase “therapeutically effective amount” refers to an amount of an antigen or vaccine that would induce an immune response in a subject receiving the antigen or vaccine which is adequate to prevent or reduce signs or symptoms of disease, including adverse health effects or complications thereof, caused by infection with a pathogen, such as a virus or a bacterium. Humoral immunity or cell-mediated immunity or both humoral and cell-mediated immunity may be induced. The immunogenic response to a vaccine may be evaluated indirectly through measurement of antibody titers, lymphocyte proliferation assays, or directly through monitoring signs and symptoms after challenge with wild type strain. The protective immunity conferred by a vaccine can also be evaluated by measuring reduction in clinical signs such as mortality, morbidity, overall physical condition, and overall health and performance of the subject.

The term “treating” refers to preventing a disorder, condition, or disease to which such term applies, or to preventing or reducing one or more symptoms of such disorder, condition, or disease.

The term “treatment” refers to the act of “treating” as defined above.

The term “vaccine” refers to a composition that elicits protective immunity in the subject.

“Protecting against infection caused by M. viscosa” refers to reduction or elimination of at least one clinical sign caused by M. viscosa. Said clinical signs include skin ulcers that may be followed by terminal septicemia and the combination thereof. In a particularly preferred embodiment, the protection against infection caused by M. viscosa refers to reduction in mortality rates caused by M. viscosa.

“Variant M. viscosa”: as noted previously, it has been determined that classic isolates (both viscous and non-viscous) of M. viscosa have a conserved sequence in their respective gyrB genes. Accordingly, classic isolates are the isolates that contain, in their respective gyrB sequences, a subsequence that is at least 98% identical to SEQ ID NO: 1. Conversely, in variant M. viscosa isolates, the respective subsequences of the gyrB sequences are less than 98% identical to SEQ ID NO: 1. Preferably, in variant M. viscosa isolates, the respective subsequences are 70-98% identical to SEQ ID NO: 1. In addition to the differences in gyrB sequences, for the purpose of this application, variant M. viscosa isolates are not recognized by antibodies raised in salmon against classic viscous M. viscosa strains under conditions described in Example 1.

In certain embodiments, the variant M. viscosa isolates have the subsequence in their gyrB gene sequences that are at least 90% identical to SEQ ID NO: 2, preferably, at least 95% identical to SEQ ID NO: 2, provided that these subsequences are no more than 98% identical to SEQ ID NO: 1.

M. viscosa

The inventors have surprisingly discovered that the antigens from a variant strain of M. viscosa cross-protect against the challenge with a classic non-viscous strain of M. viscosa and vice versa: the antigens from a classic non-viscous strain of M. viscosa cross-protect against the challenge with a variant strain of M. viscosa.

Accordingly, in the first aspect this application provides a vaccine comprising an antigenic M. viscosa component, said antigenic M. viscosa component comprising an antigen derived from a classic non-viscous M. viscosa strain, for use in protecting fish against infection caused by variant M. viscosa. Also disclosed is a vaccine comprising an antigenic M. viscosa component, said antigenic M. viscosa component comprising an antigen derived from a classic non-viscous M. viscosa strain, for use in protecting fish against infection caused by variant M. viscosa, wherein said vaccine does not contain any variant M. viscosa antigens. These vaccines may be used to protect against the infections caused by variant M. viscosa and classic non-viscous M. viscosa. In some of these vaccines, the antigenic M. viscosa component consists of the antigen derived from a classic non-viscous M. viscosa strain.

The vaccines according to this first aspect may be combined with an antigen derived from classic viscous M. viscosa. Such vaccines can be used against M. viscosa infections, including classic non-viscous M. viscosa, variant M. viscosa, and classic viscous M. viscosa.

Alternatively, the vaccines containing antigenic M. viscosa component, said antigenic M. viscosa component comprising (or consisting of, as described above) an antigen derived from a classic non-viscous M. viscosa strain may be co-administered with a vaccine containing an antigen derived from the classic viscous strain of M. viscosa. This combination of co-administered vaccines may be used for protecting fish in need thereof against infection caused by classic non-viscous M. viscosa, variant M. viscosa, and classic viscous M. viscosa.

The antigens derived from a classic non-viscous strain of M. viscosa may be provided in the form of an inactivated classic non-viscous M. viscosa preparation, such as inactivated whole organisms. Methods of bacterial inactivation are well known and include, without limitations, incubation with formalin, BEI and/or betapropiolactone (BPL). Alternatively, the antigens may be subunits, whole-cell extracts of classic non-viscous M. viscosa, or fractions thereof, including, without limitation, membrane fraction.

Similarly, antigens derived from a classic viscous strain of M. viscosa may be inactivated classic viscous M. viscosa preparation, such as inactivated whole organisms. Methods of bacterial inactivation are well known and include, without limitations, incubation with formalin, BEI and/or betapropiolactone (BPL). Alternatively, the antigens may be subunits, whole-cell extracts of classic viscous M. viscosa, or fractions thereof, including, without limitation, membrane fraction.

The antigens derived from a classic viscous strain of M. viscosa and/or a classic non-viscous strain of M. viscosa may be provided in the form of attenuated bacteria. Methods of making live attenuated bacteria are well known in the art and include, without limitation, culture passaging.

The dosage of antigenic classic non-viscous M. viscosa component and classic viscous M. viscosa component in the vaccine may vary. Thus, for example, one dose of the vaccine may contain at least 1×10⁶ cells/dose of classic non-viscous M. viscosa component. Without limitations, one dose may contain about 5×10⁶ cells/dose, about 1×10⁷ cells/dose, about 5×10⁷ cells/dose, about 1×10⁸ cells/dose, 3×10⁸ cells/dose, 5×10⁸ cells/dose, 1×10⁹ cells/dose. The dose may also contain from 1×10⁶ cells/dose to 1×10⁷ cells/dose, or 5×10⁶ cells/dose to 5×10⁷ cells/dose, or 1×10⁷ cells/dose to 1×10⁸ cells/dose, or 5×10⁷ cells/dose to 5×10⁸ cells/dose, or 1×10⁸ cells/dose to 1×10⁹ cells/dose.

Similarly, the amount of antigenic classic viscous M. viscosa component present in one dose of the vaccine (whether the same vaccine as the vaccine containing said antigenic variant M. viscosa component or the second vaccine) may be at least 1×10⁶ cells/dose of classic viscous M. viscosa component. Thus, for example, one dose of the vaccine may contain at least 1×10⁶ cells/dose of classic viscous M. viscosa component. Without limitations, one dose may contain about 5×10⁶ cells/dose, about 1×10⁷ cells/dose, about 5×10⁷ cells/dose, about 1×10⁸ cells/dose, 3×10⁸ cells/dose, 5×10⁸ cells/dose, 1×10⁹ cells/dose. The dose may also contain from 1×10⁶ cells/dose to 1×10⁷ cells/dose, or 5×10⁶ cells/dose to 5×10⁷ cells/dose, or 1×10⁷ cells/dose to 1×10⁸ cells/dose, or 5×10⁷ cells/dose to 5×10⁸ cells/dose, or 1×10⁸ cells/dose to 1×10⁹ cells/dose.

In the second aspect this application provides a vaccine comprising an antigenic M. viscosa component, said antigenic M. viscosa component comprising an antigen derived from a variant M. viscosa strain, for use in protecting fish against infection caused by classic non-viscous M. viscosa. Also disclosed is a vaccine comprising an antigenic M. viscosa component, said antigenic M. viscosa component comprising an antigen derived from a variant M. viscosa strain, for use in protecting fish against infection caused by classic non-viscous M. viscosa, wherein said vaccine does not contain any classic non-viscous M. viscosa antigens. These vaccines may be used to protect against the infections caused by classic non-viscous M. viscosa and variant M. viscosa. In some of these vaccines, the antigenic M. viscosa component consists of the antigen derived from a variant M. viscosa strain.

The vaccines according to this second aspect may be combined with an antigen derived from classic viscous M. viscosa. Such vaccines can be used against M. viscosa infections, including variant M. viscosa, classic non-viscous M. viscosa, and classic viscous M. viscosa.

Alternatively, the vaccines containing antigenic M. viscosa component, said antigenic M. viscosa component comprising (or consisting of, as described above) an antigen derived from a variant M. viscosa strain may be co-administered with a vaccine containing an antigen derived from the classic viscous strain of M. viscosa. This combination of co-administered vaccines may be used for protecting fish in need thereof against infection caused by variant M. viscosa, classic non-viscous M. viscosa, and classic viscous M. viscosa.

The antigens derived from a variant strain of M. viscosa may be inactivated variant M. viscosa preparation, such as inactivated whole organisms. Methods of bacterial inactivation are well known and include, without limitations, incubation with formalin, BEI and/or betapropiolactone (BPL). Alternatively, the antigens may be whole-cell extracts of variant M. viscosa, or fractions thereof, including, without limitation, membrane fraction.

Similarly, antigens derived from a classic viscous strain of M. viscosa may be an inactivated classic viscous M. viscosa preparation, such as inactivated whole organisms. Methods of bacterial inactivation are well known and include, without limitations, incubation with formalin, BEI and/or betapropiolactone (BPL). Alternatively, the antigens may be whole-cell extracts of classic viscous M. viscosa, or fractions thereof, including, without limitation, membrane fraction.

The antigens derived from classic viscous strain of M. viscosa and/or a variant strain of M. viscosa may be provided in the form of attenuated bacteria. Methods of making live attenuated bacteria are well known in the art and include, without limitation, culture passaging.

The dosage of antigenic variant M. viscosa component and classic viscous M. viscosa component in the vaccine may vary. Thus, for example, one dose of the vaccine may contain at least 1×10⁶ cells/dose of the variant M. viscosa component. Without limitations, one dose may contain about 5×10⁶ cells/dose, about 1×10⁷ cells/dose, about 5×10⁷ cells/dose, about 1×10⁸ cells/dose, 3×10⁸ cells/dose, 5×10⁸ cells/dose, 1×10⁹ cells/dose. The dose may also contain from 1×10⁶ cells/dose to 1×10⁷ cells/dose, or 5×10⁶ cells/dose to 5×10⁷ cells/dose, or 1×10⁷ cells/dose to 1×10⁸ cells/dose, or 5×10⁷ cells/dose to 5×10⁸ cells/dose, or 1×10⁸ cells/dose to 1×10⁹ cells/dose.

Similarly, the amount of antigenic classic viscous M. viscosa component present in one dose of the vaccine (whether the same vaccine as the vaccine containing said antigenic classic non-viscous M. viscosa component or the second vaccine) may be at least 1×10⁶ cells/dose of classic viscous M. viscosa component. Thus, for example, one dose of the vaccine may contain at least 1×10⁶ cells/dose of classic viscous M. viscosa component. Without limitations, one dose may contain about 5×10⁶ cells/dose, about 1×10⁷ cells/dose, about 5×10⁷ cells/dose, about 1×10⁸ cells/dose, 3×10⁸ cells/dose, 5×10⁸ cells/dose, 1×10⁹ cells/dose. The dose may also contain from 1×10⁶ cells/dose to 1×10⁷ cells/dose, or 5×10⁶ cells/dose to 5×10⁷ cells/dose, or 1×10⁷ cells/dose to 1×10⁸ cells/dose, or 5×10⁷ cells/dose to 5×10⁸ cells/dose, or 1×10⁸ cells/dose to 1×10⁹ cells/dose.

The inventors have also surprisingly discovered that vaccination with variant M. viscosa antigen provides cross-protection against classic viscous M. viscosa challenge, but not the other way around (i.e., vaccination with the classic viscous M. viscosa antigen does not protect against variant M. viscosa challenge). Accordingly, in the third aspect, the invention provides a vaccine comprising an antigenic M. viscosa component, said antigenic M. viscosa component comprising an antigen derived from a variant M. viscosa strain, for use in protecting fish against infection caused by classic viscous M. viscosa.

As discussed above, the antigen derived from a variant M. viscosa strain can also be used in protecting fish against infection caused by classic non-viscous M. viscosa. Accordingly, this disclosure also provides a vaccine an antigenic M viscosa component, said antigenic M viscosa component comprising an antigen derived from a variant M viscosa strain, for use in protecting fish against infection caused by classic viscous M. viscosa and against infection caused by classic non-viscous M. viscosa strains. Therefore, the disclosure also provides a vaccine an antigenic M. viscosa component, said antigenic M. viscosa component comprising an antigen derived from a variant M. viscosa strain, for use in protecting fish against infection caused by classic viscous M. viscosa and against infection caused by classic non-viscous M. viscosa strains, wherein said antigenic M. viscosa component lacks antigens derived from classic viscous and classic non-viscous M. viscosa strains.

In the fourth aspect, the invention provides a vaccine comprising an antigenic M. viscosa component, said antigenic M. viscosa component comprising an antigen derived from a classic non-viscous M. viscosa strain, for use in protecting fish against infection caused by classic viscous M. viscosa.

As discussed above, the antigen derived from a classic non-viscous M. viscosa strain can also be used in protecting fish against infection caused by variant M. viscosa. Accordingly, this disclosure also provides a vaccine an antigenic M. viscosa component, said antigenic M. viscosa component comprising an antigen derived from a classic non-viscous M. viscosa strain, for use in protecting fish against infection caused by classic viscous M. viscosa and against infection caused by variant M. viscosa strains. Therefore, the disclosure also provides a vaccine an antigenic M. viscosa component, said antigenic M. viscosa component comprising an antigen derived from a classic non-viscous M. viscosa strain, for use in protecting fish against infection caused by classic viscous M. viscosa and against infection caused by variant M. viscosa strains, wherein said antigenic M. viscosa component lacks antigens derived from classic viscous and variant M. viscosa strains.

The dosages of classic viscous, classic non-viscous and variant M. viscosa antigens for the vaccines described in connection with the first and the second aspects are also applicable for the vaccines according to these third and the fourth aspects of invention.

### Additional Antigens

In all four aspects, the respective antigenic components of the vaccines (or the second vaccines) described herein may contain one or more additional, non-M. viscosa antigens as described below.

Such antigens may be derived from a bacterial source, from a viral source, from an additional parasitical source, and/or from a fungal source. These additional antigens may be inactivated organisms recited below, or the antigens may be derived from these organisms, including recombinantly prepared antigens.

Polyvalent vaccines containing antigens from typical fish pathogens other than M. viscosa are well known in the art and are already commercially available. In addition, representative isolates of relevant fish pathogens are available from various sources.

In particular embodiments of the invention said antigen from a bacterial source is selected from the group consisting of: live, attenuated or killed bacteria of the species Piscirickettsias sp. Aeromonas sp., Vibrio sp., Aliivibrio sp., Listonella sp., Tenacibaculum sp., Pasteurella sp., Photobacterium sp, Flavobacterium sp., Yersinia sp., Renibacterium sp., Streptococcus sp., Lactococcus sp., Leuconostoc sp., Bifidobacterium sp., Pediococcus sp., Brevibacterium sp., Edwarsiella sp., Francisella sp., Pseudomonas sp., Cytophaga sp., Nocardia sp., Mycobacterium sp., parts or subunits of these bacteria, and any combination hereof.

Isolates of such bacteria are available, e.g. from LGC Promochem/American Type Culture Collection ATCC repository and distribution center (ATCC) including strains of A. salmonicida (ATCC 33658), V. salmonicida (ATCC 43839), V. anguillarum serotype 01 (ATCC 43305) and O2(ATCC 19264). In addition, cultures of Piscirickettsias salmonis have been deposited in the European Collection of Cell Culture (ECACC), Health Protection Agency, Porton Down, Salisbury, Wiltshire (UK), SP4 0JG UK on the 9 Jun. 2006 under the following accession numbers: 06050901, 06050902, 06050903 and 07032110.

Other specific embodiments pertain to a vaccine, wherein said antigenic material obtained from a viral source other than the fish virus as defined above is from a virus selected from the group consisting of: Viral Hemorrhagic Septicemia Virus (VHSV), Infectious Hematopoietic Necrosis virus (IHNV), Infectious Pancreatic Necrosis Virus (IPNV), Infectious Salmon Anaemia virus (ISAV), Salmon pancreatic disease virus (SPDV), Iridovirus, Nodavirus, Piscine myocarditis virus (PMCV) and heart and skeletal muscle inflammation virus (HSMIV). These antigens may be included as modified live or inactivated organisms, as parts or subunits of any one of these viruses, as DNA vaccines, and/or combinations thereof. Representative species of such viruses are available to the skilled artisan, for instance from the following deposits: infectious pancreatic necrosis virus (IPNV, ATCC VR-1318, country of origin: unknown), Viral Hemorrhagic Septicemia Virus (VHSV, ATCC VR_1389, country of origin: Denmark); Infectious Hematopoietic Necrosis virus (IHNV, ATCC VR-1392, country of origin: USA)); Pancreatic Necrosis Virus; Infectious Salmon Anaemia (ISA) virus (ATCC VR-1554, country of origin: Canada).Patent deposits have previously been made by the present applicant of the following viral species: Heart and Skeletal Muscle Infection Virus (HSMIV, patent deposit nr ECACC 04050401, country of origin: Norway).

In more specific embodiments, said antigenic material obtained from a viral source other than the fish virus as defined above is from the group consisting of: Glycoprotein of Viral Hemorrhagic Septicemia Virus (VHSV), nucleoprotein of Viral Hemorrhagic Septicemia Virus (VHSV), glycoprotein of Infectious Hematopoietic Necrosis virus (IHNV), nucleoprotein structural proteins of Infectious Pancreatic Necrosis Virus (IPNV), antigenic fragments of any of one of these proteins and combinations hereof.

In other embodiments said antigenic material from an additional parasitic source is from a source selected from the Lepeophtheirus Sp., Caligus Sp., and Ichthyophthirius Sp, parts of any one of these parasites, and combinations thereof. In yet other embodiments said antigenic material is from a fungal source selected from the group consisting of Saprolegnia Sp., Branchiomyces sanguinis, Branchiomyces demigrans and Icthyophonus hoferi.

In certain embodiments, the additional antigens to be included into the vaccine of the invention and/or the second vaccine containing the classic viscous M. viscosa are selected form the group consisting of IPNV, ISAV, SPDV, Aeromonas salmonicida, Vibrio anguillarum 01, 02, Vibrio (Aliivibrio) salmonicida, Yersinia ruckeri 01.

In other embodiments, the additional antigens to be included into the vaccine of the invention and/or the second vaccine containing the classic viscous M. viscosa are selected form the group consisting of IPNV, Aeromonas salmonicida, Vibrio anguillarum serotype 1 and 02, and Vibrio (Aliivibrio) salmonicida.

### Excipients and Adjuvants

The vaccines of the invention may further comprise a suitable pharmaceutical carrier and/or an adjuvant. The pharmaceutical carriers can be sterile liquids, such as water or buffer solutions, such as saline solutions and aqueous dextrose and glycerol solution. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. Examples of suitable pharmaceutical carriers are described in “Remington's Pharmaceutical Sciences” by E. W. Martin. The formulation should suit the mode of administration.

The appropriate carrier is evident to those skilled in the art and will depend in large part upon the route of administration. Additional components that may be present in this invention are adjuvants, preservatives, surface active agents, chemical stabilizers, suspending or dispersing agents. Typically, stabilizers, adjuvants and preservatives are optimized to determine the best formulation for efficacy in the target subject.

In a currently preferred embodiment, the vaccine comprises an adjuvant. Suitable adjuvants include, without limitations, oil. The vaccines disclosed herein may be formulated as oil in water emulsions or, more preferably, water-in-oil emulsions. Other formulations, such as water-in-oil-in-water (W/O/W) may also be prepared. In addition, the vaccine may comprise one or more suitable surface-active compounds or emulsifiers, e.g. CREMOPHORE®, TWEEN® and SPAN®. Also adjuvants such as interleukin, CpG and glycoproteins may be used.

The vaccine may also comprise a “vehicle”. A vehicle is a device to which the antigen adheres, without being covalently bound to it. Such vehicles are biodegradable nano/micro-particles or -capsules of PLGA (poly-lactide-co-glycolic acid), alginate or chitosan, liposomes, niosomes, micelles, multiple emulsions and macrosols, all known in the art. A special form of such a vehicle, in which the antigen is partially embedded in the vehicle, is the so-called ISCOM (European patents EP 109.942, EP 180.564 and EP 242.380.

In certain embodiments, the vaccine described herein is formulated as a water-in-oil emulsion. Preferably, the oil is a mineral oil.

The vaccines described herein may be administered to the salmonid by a variety of routes, including, without limitation, intraperitoneally, intramuscularly, orally, and by immersion. Preferably, the vaccine is administered by an injection in a microdose such that the volume of one dose is under 500 μl, or under 400 μl, or under 300 μl or under 200 μl or about 100 μl or under 100 μl, or about 50 μl or about 25 μl.

The vaccine disclosed herein may be used in protecting multiple salmonid species against an infection. Suitable salmonids include, without limitations, Atlantic salmon (Salmo salar), coho salmon (Oncorhynchus kisutch), rainbow trout (Oncorhynchus mykiss), sockeye salmon (Oncorhynchus nerka), Chinook salmon (Oncorhynchus tshawytscha) and other species.

Salmonids of different ages (or weights) may be vaccinated according to the invention. In certain embodiments, the salmonid weighs between about 15 and about 200 grams at the time of vaccination. Thus, the weight of the salmonid at the time of the vaccination may be between about 25 and about 150 grams or between about 40 and about 110 grams or between about 50 and about 100 grams.

The invention will now be described in the following illustrative examples.

### EXAMPLES

### Example 1: Variant and Classic Non-Viscous M. viscosa are not Recognized by a Salmon Polyclonal Antibody Raised Against Classic Viscous M. viscosa

### Materials and Methods Western Blot:

Ten strains of M. viscosa were investigated using Western blot. Origin and year of isolation, gyrB variant or classic type as well as viscosity phenotype with regards to viscosity are listed in table 1. Isolate number also corresponds to lane number in the Western blot membranes.

TABLE 1
Overview of M. viscosa strains included in the
study. The bacteria were not inactivated prior
to sample preparation under reducing conditions.

| Isolate | | | | gyrB (classic | Viscous |
|---|---|---|---|---|---|
| number | Country | Species | Year | or variant)^{#} | (yes/no)* |
| 1 | Norway | Atlantic | 2018 | variant | Yes |
| | | salmon | | | |
| 2 | Norway | Atlantic | 2020 | variant | Yes |
| | | salmon | | | |
| 3 | Norway | Atlantic | 2001 | classic | Yes |
| | | salmon | | | |
| 4 | Norway | Atlantic | 2020 | classic | Yes |
| | | salmon | | | |
| 5 | Norway | Atlantic | 2021 | classic | No |
| | | salmon | | | |
| 6 | Norway | Atlantic | 2020 | classic | No |
| | | salmon | | | |
| 7 | Norway | Rainbow | 2019 | variant | Yes |
| | | trout | | | |
| 8 | Norway | Lumpfish | 2019 | variant | Yes |
| 9 | Canada | Atlantic | 2019 | variant | Yes |
| | | salmon | | | |
| 10 | Iceland | Atlantic | 2021 | variant | Yes |
| | | salmon | | | |

*When grown on blood agar at 15° C. for 48 hours at 2% NaCl.
^{#}Adapted from Grove et al., Dis Aquat Org, 93: 51-61,2010.

### Antibodies/Conjugates Used:

1) Monoclonal mouse anti-trout/salmon IgM antibody, clone 4C10
2) Polyclonal rabbit anti-mouse HRP conjugated (Cat. #P0260, Dako)
3) Precision Protein STREPTACTIN® HRP conjugated (Cat. #161-0380, Bio Rad)
4) Polyclonal salmon a-variant M. viscosa antibody-generated in course of the project
5) Polyclonal salmon a-classic M. viscosa antibody-generated in course of the project
6) Polyclonal rabbit anti-variant M. viscosa antibody-generated in course of the project
7) Polyclonal rabbit anti-classic M. viscosa antibody-generated in course of the project
8) Swine anti-rabbit HRP conjugated (Cat. #P0217, Dako)

TABLE 2
List of materials.
Materials

Tris-buffered Saline + Tween (TBST)
Tris-buffered Saline (TBS)
Sample buffer:
2x Laemmli Sample Buffer, Bio-Rad
2-Mercaptoethanol, Sigma-Aldrich (diluted 1:20 in 2x Laemmli Sample
Buffer)
CLARITY ™ Western ECL Substrate, Bio-Rad
PRECISION PLUS PROTEIN ™ All Blue Standards, Bio-Rad
PRECISION PLUS PROTEIN ™ Unstained Standards, Bio-Rad
CRITERION ™ TGX Stain-Free Precast Gel 4-20%, Bio-Rad
TRANS-BLOT TURBO BLOT ™, Bio-Rad
Skim milk powder, Millipore

### Sample Preparation:

Bacterial cultures were spread on blood agar plates for each strain. The plates were incubated at 15° C. After two days incubation, colonies from the agar plates were inoculated in 10 ml growth medium in 25 cm² cell flasks and incubated at 15° C. and 100 rpm.

After incubation, OD was measured. 2×1 ml were centrifuged, the supernatant pipetted out.

The 10 bacterial pelleted samples were prepared for Western blot. All pellets were resolved in reducing sample buffer, incubated at 100° C. for 10 minutes and frozen until further analyses. The day of SDS-PAGE the samples were diluted/normalized to obtain a theoretical OD 2 (calculated from OD at harvest) with freshly made sample buffer, before they were used.

### SDS-PAGE and WB:

10 μl of each sample were added to the lanes on the gel (6 μl in the molecular weight marker-lanes). Four parallel gels were run.

The gels were run for approx. 3 minutes at 250 V and then approximately 50 minutes at 160 V. Equal volume of PRECISION PLUS PROTEIN™ All Blue Standard and PRECISION PLUS PROTEIN™ Unstained Standard were combined (3-5 μl of each) and used as a molecular weight standard. Four gels were made to make four blots; two for treatment with salmon plasmas and two with rabbit anti-M. viscosa antibodies. The gels were then activated for 45 seconds on the gel-doc, imaged and transferred to the TRANS-BLOT® TURBO™ Transfer Blotting System (BioRad), where a 7-minute turbo program was used. The blotted membranes were immediately transferred to a blocking-buffer (5% skimmed milk in TBST (TBSTM)). The blots were blocked over night at 2-6° C.

The blots were then incubated with antibodies as described in table 3 and 4. The STREPTACTIN® HRP was added together with the other HRP-conjugated antibodies for visualisation of the molecular weight standards. The blots were washed 3×10 minutes with TBST between each incubation (TBS was used in the last washing step before substrate incubation) before incubation with the CLARITY™ Western substrate for 5 minutes before exposure on the gel-doc imaging system (BioRad).

TABLE 3
Antibodies, dilutions and incubation times for blots incubated
with salmon antibodies. RT = Room temperature

| Antibody | | Dilution | Incubation |
|---|---|---|---|
| Ab1 | Blot 1: Polyclonal salmon a- variant | 1:2000 | Overnight. |
| | M. viscosa antibody | | 2-8° C. |
| | Blot 2: Polyclonal salmon a-classic | | |
| | M. viscosa antibody | | |
| Ab2 | Monoclonal mouse anti-trout/salmon | 1:3500 | 1 h, RT. |
| | IgM antibody, clone 4C10 | | |
| Ab3 | Rabbit anti-mouse- HRP conjugate, DAKO | 1:1000 | 1 h, RT. |
| | StrepTactin -HRP conjugate, BioRad | 1:3000 | |

TABLE 4
Antibodies, dilutions and incubation times for blots incubated
with rabbit antibodies. RT = Room temperature

| Antibody | | Dilution | Incubation |
|---|---|---|---|
| Ab1 | Blot 3: Polyclonal rabbit anti-variant | 1:1000 | 2 h, RT. |
| | M. viscosa antibody | | |
| | Blot 4: Polyclonal rabbit anti-classic | | |
| | M. viscosa antibody | | |
| Ab2 | Swine anti-rabbit -HRP conjugate, DAKO | 1:1000 | 1 h, RT. |
| | StrepTactin -HRP conjugate, BioRad | 1:3000 | |

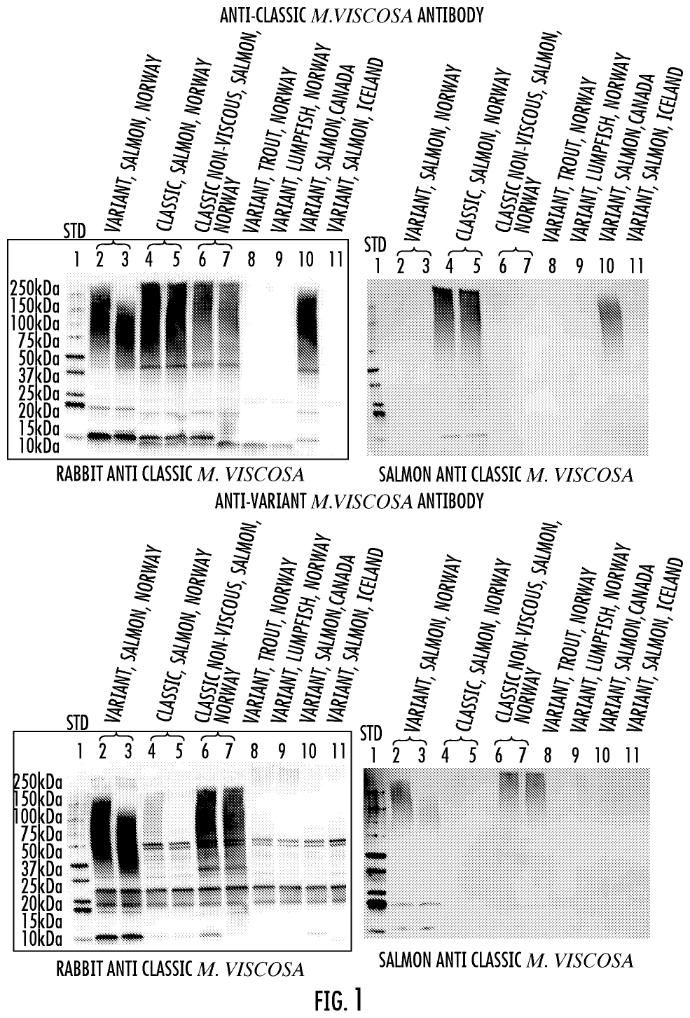

The results of the experiment are illustrated in FIG. 1. This figure demonstrates that classic viscous M. viscosa and variant M. viscosa are recognized by different antibodies. More specifically, these results show that the antibody raised against classic viscous M viscosa in salmon does not recognize classic non-viscous M. viscosa and variant M. viscosa. Surprisingly, it was also demonstrated that classic non-viscous M. viscosa and variant M. viscosa are recognized by the same antibody.

### Example 2: Vaccination with Variant M. Viscosa Cross-Protects Against Classic Non-Viscous Challenge, while Vaccination with Classic Viscous M. Viscosa does not

### Materials and Methods

Bath challenge studies were comparative, investigator-blinded, negative controlled and randomised laboratory experiments. The efficacy of different oil-adjuvanted injectable vaccines in protecting Atlantic salmon against experimental infection with different isolates of M. viscosa were investigated in the study. The experimental vaccines containing field isolates of classic non-viscous M. viscosa and variant M. viscosa adjuvanted with water-in-oil (W/O) emulsion were administered intraperitoneally by co-injection with commercial vaccines according to manufacturers' instructions. Both of these commercial vaccines contained classic viscous strains of M. viscosa. A negative control group was included. The fish was approximately 25 grams in average at vaccination.

The fish was exposed to continuous light (24:0) in order to smoltify prior to transfer and challenge in sea water. Onset of photomanipulation started approximately 6 weeks prior to challenge with a classic non-viscous strain of M. viscosa. Challenge was conducted by immersion 13 weeks post vaccination. Due to biomass considerations, the fish was challenged in two 500 L tanks per challenge isolate and the results from the identical replicate tanks was combined. A total of approximately 60 fish (30 fish/group per tank) per group was challenged per challenge isolate.

One week prior to challenge, the fish was transferred to the disease facility and equally distributed into two duplicate 500 L tanks per challenge isolate and adapted to 34% salinity seawater, 8° C., 24:0 light regimen, which was the environmental parameters during the challenge period. The challenge material was cultivated fresh from a frozen bacterial stock, in shake flask culture medium based on yeast extract at 12 degrees Celsius for two days with shaking. The fish was challenged by reducing the water volume in the tank before adding the challenge material directly into the tank.

Challenge was performed 13 weeks post vaccination. The fish were observed daily post challenge, and fish with ulcers were euthanized and recorded as mortalities in the mortality log. Efficacy was evaluated by statistical comparison of protection against mortality and ulceration between the vaccinated groups and the negative control group post challenge. The study fish were unvaccinated, free from clinical disease and had a valid health certificate. Injured or deformed fish were excluded from the study.

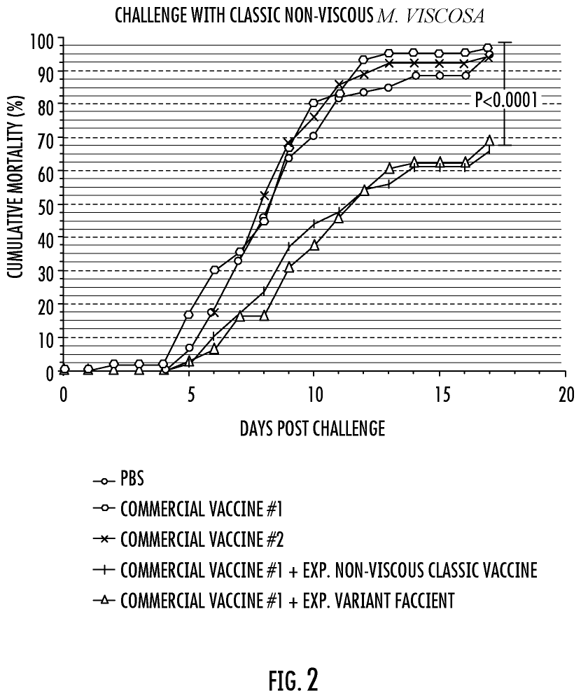

The results of the experiment are illustrated in FIG. 2.

As one can see, the group treated with PBS as well as the groups treated with commercial vaccines (both containing classic viscous M. viscosa antigen) resulted in almost 100% mortality eighteen days after the fish was challenged with a classic non-viscous M. viscosa strain, thus supporting the validity of the challenge model. There was no statistically significant difference between these three groups.

In contrast, groups vaccinated with compositions comprising either a classic non-viscous M. viscosa antigen or a variant antigen exhibited only about 70% mortality (no significant difference between these two groups but p<0.0001 compared to the groups treated with PBS or commercial vaccines).

These results suggest that classic viscous M. viscosa antigens do not cross-protect against the challenge with classic non-viscous M. viscosa strains. Classic non-viscous antigens protect against the challenge with classic non-viscous M. viscosa strains. Surprisingly, variant M. viscosa antigens cross-protect against the challenge with classic non-viscous M. viscosa strains.

### Example 3: Vaccination Classic Non-Viscous M. viscosa Cross-Protects Against Variant M. viscosa Challenge, while Vaccination with Classic Viscous M. viscosa does not

The materials and methods were the same as in Example 2, except a variant M. viscosa isolate was used as a challenge strain.

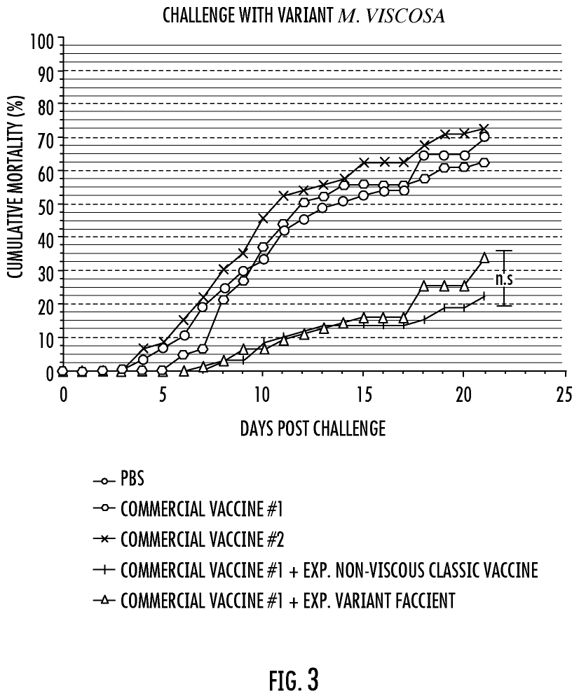

The results of the experiment are illustrated in FIG. 3.

As one can see, the group treated with PBS as well as the groups treated with commercial vaccines (both containing classic viscous M. viscosa antigen) resulted in about 60 to 70% mortality 22 days after the fish was challenged with a classic non-viscous M. viscosa strain, demonstrating that the challenge model was valid. There was no statistically significant difference between these three groups.

In contrast, groups vaccinated with compositions comprising either a classic non-viscous M. viscosa antigen or a variant antigen exhibited only about 20-35% mortality (no significant difference between these two groups but p<0.0001 compared to the groups treated with PBS or commercial vaccines).

These results suggest that classic viscous M. viscosa antigens do not cross-protect against the challenge with variant M. viscosa strains. Variant antigens protect against the challenge with variant M. viscosa strains. Surprisingly, classic non-viscous M. viscosa antigens cross-protect against the challenge with variant M. viscosa strains.

### Example 4: Vaccination with Variant M. Viscosa Protects Against the Challenge with Classic Viscous M. Viscosa

### Materials and Methods

The cross-protective efficacy of the monovalent oil-adjuvanted (W/O emulsion) variant M. viscosa vaccine was assessed in an investigator-blinded, negative controlled and randomised laboratory experiment.

A monovalent variant M. viscosa vaccine was administered by intraperitoneal injection to one group. A second group was vaccinated with a vaccine containing classic viscous M. viscosa and a negative control group was injected with Phosphate Buffered Saline (PBS). The fish were kept in 500 L tanks at 15° C. freshwater during the immunisation period, and were exposed to a continuous light regime (24:0 light:dark) for approximately 6 weeks in order to smoltify prior to bath challenge in seawater. The challenge material (a classic viscous M. viscosa isolate) was cultivated fresh from a frozen bacterial stock, in shake flask culture medium based on yeast extract at 12 degrees Celsius for two days with shaking. The fish was challenged by reducing the water volume in the tank before adding the challenge material directly into the tank.

Challenge was performed by immersion after an immunisation period for approximately 9 weeks. The challenge intended to investigate any cross-protective efficacy of the monovalent vaccine containing inactivated antigen of variant M. viscosa against a classic viscous M. viscosa isolate. Approximately one week prior to challenge, the fish were transferred to the disease facility and the groups were equally distributed into parallel 500 L tanks and gradually acclimatized to 8° C., 34‰ salinity seawater. In order to reduce biomass density at challenge, the groups were challenged in two duplicate tanks per challenge isolate, and the results from the two tanks were combined. The fish were observed daily post challenge, and fish with ulcers were euthanized and recorded as mortalities in the mortality log. Efficacy was evaluated by statistical comparison of protection against mortality and ulceration between the vaccinated groups and the negative control group post challenge. The study fish were unvaccinated, free from clinical disease and had a valid health certificate. Injured or deformed fish were excluded from the study.

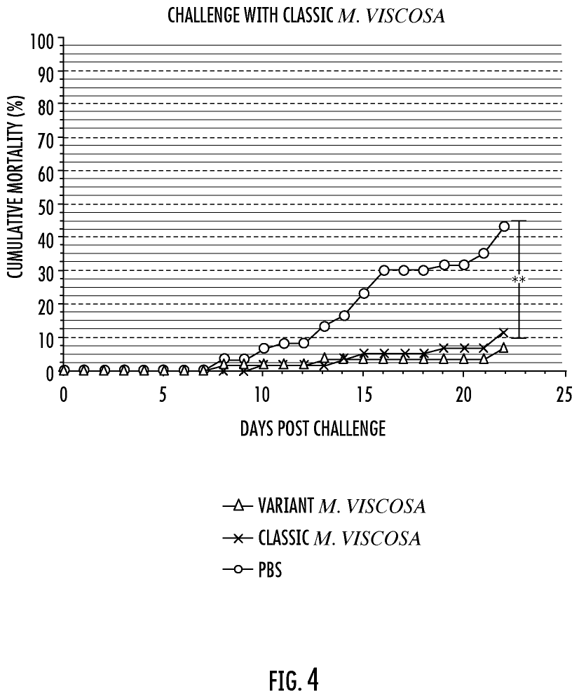

The results are illustrated in FIG. 4. The group treated with PBS exhibited over 40% mortality 22 days after the challenge. In contrast, vaccination with a classic M. viscosa antigen or with a variant M. viscosa antigen resulted in a statistically significant drop in cumulative mortality, to about 10 and 75, respectively, by day 22 after the challenge. These results indicate that vaccination with a variant M. viscosa antigen can cross-protect against the challenge with a classic viscous M. viscosa. Given the similar responses of the groups vaccinated with variant M. viscosa antigens and the groups vaccinated with classic-non-viscous M. viscosa antigens, these results also strongly suggest that vaccination with a classic non-viscous M. viscosa antigen can cross-protect against the challenge with a classic M. viscosa.

All publications cited in the specification, both patent publications and non-patent publications, are indicative of the level of skill of those skilled in the art to which this invention pertains. All these publications are herein fully incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the following claims.

1-39. (canceled)

40. A method of protecting fish in need thereof against infection caused by classic non-viscous M. viscosa, the method comprising administering to said fish a vaccine comprising an antigenic M. viscosa component, said antigenic M. viscosa component comprising an antigen derived from a variant M. viscosa.

41. The vaccine of claim 40, wherein said fish is protected against infection caused by variant M. viscosa and by classic non-viscous M. viscosa.

42. The vaccine of claim 40, wherein said vaccine does not include an antigen derived from a classic non-viscous M. viscosa strain.

43. The method of claim 40, wherein said antigenic M. viscosa component consists of the antigen derived from the variant M. viscosa strain.

44. The method of claim 40, wherein said vaccine is for use to be co-administered with a second vaccine comprising an antigen derived from a classic viscous M. viscosa strain.

45. The method of claim 44, wherein said second vaccine does not contain an antigen derived from a classic non-viscous M. viscosa strain.

46. The method of claim 40, wherein the antigenic M. viscosa component consists of the antigen derived from the variant M. viscosa strain.

47. The method of claim 40, wherein the antigen derived from the variant M. viscosa strain is an inactivated preparation of said variant M. viscosa strain.

48. The method according to claim 40, comprising one or more non M. viscosa antigens.

49. The method of claim 48 wherein said one or more non M. viscosa antigens are selected from the group consisting of IPNV, ISAV, SPDV, Aeromonas salmonicida, Vibrio anguillarum O1, O2, Vibrio (Aliivibrio) salmonicida, Yersinia ruckeri O1.

50. The method of claim 40 wherein said fish is a salmonid.

51. The method of claim 50 wherein said salmonid is Atlantic salmon (Salmo salar).

52. The method of claim 40 wherein said vaccine is administered peritoneally.

53. The method according to claim 40, wherein protecting said fish against infection comprises a reduction or an elimination of at least one symptom of M. viscosa.

54. The vaccine according claim 40, wherein protecting said fish against infection comprises a reduction of mortality caused by M. viscosa.


SEQUENCE LISTING (2 sequences)

SEQ ID NO: 1 - DNA, 411 bp, Moritella viscosa
  source 1..411: mol_type = genomic DNA
ttaaccattcatcgccacggtcatatccatcagcaaatttatcgtcatggtgaaccacaagcgccgatcgctgtgattggtgatattgatacaacgggtactgaattacgcttttggcctagtcatgagactttcaccaatacattatttcattatgatattttagcaaaacgtttacgtgagttatctttcctaaattcaggtgtttcaattaaattaattgatgaacgtgaagacggaaaatcggatcatttcgtttatgaaggtggtattaaggcatttgttgattacttaaacgttaacaaaacaccaatcaacaaagacgcatttcacttctctgttgaacgtgaagatggaattactgttgaagttgctatgcagtggaacgatggtttccaagaaggcatttat

SEQ ID NO: 2 - DNA, 411 bp, Moritella viscosa
  source 1..411: mol_type = genomic DNA
cttacgattttccgtcacggtaaaattcataaccaaacatatcgtcatggtgtaccagatgggccaatggctgagacaggggttatcgatggcacgggtacagagttacgtttttggcctagtcatgagaccttcactaatacattattccattacgatattttagcaaaacgtttacgtgagttatctttcctaaactcaggtgtttcaattaaattaattgatgaacgtgaagacggtaaatcggatcatttcgtttatgaaggtggcatcaaggcatttgttgattacttaaatgtaaacaaaacaccaatcaacaaagacgcgtttcacttctcggctgaacgtgaagatggcattaccgttgaagttgcgatgcagtggaacgatggtttccaagaaggcatctat